SEQUENCE LISTING (2 sequences)

SEQ ID NO: 1 - DNA, 35 bp, Homo sapiens
agggaagaaagcgaaaggaggctgccagcgacgag

SEQ ID NO: 2 - DNA, 35 bp, Homo sapiens
aaccaataggaacgccatcagctgccagcgacgag

(19) United States
(12) Patent Application Publication
(10) Pub. No.: US 2003/0124592 A1
(43) Pub. Date: Jul. 3, 2003

(54) Methods for detecting genetic haplotypes by interaction with probes

(75) Inventor: Robert S. Puskas, Manchester, MO (US)

Correspondence Address: SONNENSCHEIN NATH & ROSENTHAL, P.O. BOX 061080, WACKER DRIVE STATION, CHICAGO, IL, 60606-1080, US

(73) Assignee: BioProfile, LLC

(21) Appl. No.: 10/280,580

(22) Filed: Oct. 24, 2002

Related U.S. Application Data
(60) Provisional application No. 60/335,040, filed on Oct. 24, 2001.

(51) Int. Cl. C12Q 1/68

(52) U.S. Cl. 435/6

(57) ABSTRACT

The present invention provides methods for determining genetic haplotype comprising identifying target nucleic acids or gene fragments, said nucleic acids or gene fragments comprising a haplotype of interest, and detecting at least one parameter displayed by one of a primer-dependent transcript, a probe, or a primer-dependent transcript/probe complex. Further provided are methods for determining genetic haplotype comprising labeling nucleic acids or gene fragments with a least one probe, and detecting the velocity of the nucleic acid molecule or gene fragment by sequentially measuring at least one parameter displayed by the probes.

### CROSS-REFERENCE TO RELATED APPLICATIONS

This application claims priority from Provisional Application Serial No. 60/335,040 filed on Oct. 24, 2001, which is hereby incorporated by reference in its entirety.

### STATEMENT REGARDING FEDERALLY SPONSORED RESEARCH OR DEVELOPMENT

Not Applicable.

### REFERENCE TO A MICROFICHE APPENDIX

Not Applicable.

### REFERENCE TO A SEQUENCE LISTING

The Sequence Listing, which is a part of the present disclosure, includes a text file containing the nucleotide sequences of the present invention on a floppy disc. The subject matter of the Sequence Listing is herein incorporated by reference in its entirety.

### BACKGROUND OF THE INVENTION

1. Field of the Invention

The present invention relates to apparatus and methods for identifying genetic haplotypes by direct detection of nucleic acid fragments or molecules marked by interaction with at least one probe.

2. Description of Related Art

Investigators have identified millions of nucleotide positions where single base changes, base insertions, or base deletions may occur in the human genome. These genetic variations (GVs) in the genetic composition of an individual determine genetic diseases, predisposition to diseases, ability to metabolize therapeutics, rate of metabolism of therapeutics, side effects of therapeutics, and the like.

Typically, in samples of DNA or cDNA derived from tissues or cells that have two chromosomes (i.e., all normal somatic tissues in humans and animals) in which there are two or more heterozygous sites, it is generally impossible to tell which nucleotides belong together on one chromosome when using genotyping methods such as (i) DNA sequencing, (ii) nucleic acid hybridization of oligonucleotides to genomic DNA or total cDNA or amplification products derived therefrom, (iii) nucleic acid hybridization using probes derived from genomic DNA or total cDNA or amplification products derived therefrom, or (iv) most amplification-based schemes for variance detection.

Haplotypes can be inferred from genotypes of related individuals by using a pedigree to sort out the transmission of groups of neighboring variances, but pedigree analysis is of little or no use when unrelated individuals are the subject of investigation, as is frequently the case in medical studies. There are some methods for determining haplotypes in unrelated individuals, for example, methods based on setting up allele-specific PCR primers for each of two variances that are being scanned (Michalatos-Beloin, et al., Nucl. Acids Res. 24: 4841-4843 (1996)); however, these methods generally require customization for each locus to be haplotyped, and can therefore be time-consuming and expensive. In addition, these methods are limited to determining haplotypes for regions covering less than 20 kilobases.

Investigators also have determined that often it is not merely the presence of GVs that cause the above phenotypic variations, but rather the distribution or configuration of GVs on the chromosomes of the individual (Hess, P., et al., Impact of pharmacogenomics on the clinical laboratory, Mol. Diagn. 4:289-98 (1999); Davidson, S., Research suggests importance of haplotypes over SNPs, Nature Biotechnology 18:1134-5 (2000)). For example, two individuals may be heterozygous for three GVs in a specific region of the chromosome, but only one of the individuals will have a genetic disease because of the difference in haplotype (GV configuration) between the two individuals. In the unaffected individual two of the GVs occur on one chromosome and the other GV occurs on the other chromosome, while in the diseased individual, all three GVs occur on the same chromosome.

The ability to determine haplotypes is crucial to the investigation of genetic diseases and the development of personalized therapeutics. Current methods for detecting haplotypes are lengthy and cumbersome. Most existing methods require many steps, testing of many samples and/or the use of specific software to determine relevant haplotypes (See, e.g., U.S. Pat. No. 6,183,958 to Stanton; U.S. Pat. No. 6,235,502 to Weissman, each of which is incorporated by reference herein in its entirety).

Affymetrix described a method using chip technology that can be used to determine haplotypes. Oligonucleotide probes for two GS sites are linked to the same chip site. Hybridization of nucleic acids to both probes was detected by the increase in hybrid yield that occurs with cooperative hybridization to both probes, compared with hybridization to either of the probes separately (Gentalen, E., et al., A novel method for determining linkage between DNA sequences: hybridization to paired probe arrays, Nucleic Acids Res. 27(6):1485-91 (1999)). This method is limited to analyzing two probes per chip site. Factors also limit the method to analyzing GV sites no further apart than about 2,000 bases. Chip technologies also require significant amounts of target material for effective use. Although the target nucleic acids can be amplified prior to assay using PCR or similar amplification technologies, these additional steps increase the complexity of the assay and add steps that can be affected by contaminating materials present in a sample.

U.S. Pat. No. 5,104,791 to Abbott, et al., incorporated by reference herein in its entirety, describes a method of detecting target nucleic acids using two nucleic acid probes. One probe is a particle-bound capture probe and the second probe is reporter nucleic acid probe. Detection of target occurs via concurrent detection of particles (microspheres) and the reporter probe, which can be fluorescent, radioactive, luminescent, or the like. However, unlike the present invention, this method is limited to using nucleic acid probes. Further, the method was not conceived as a means of determining haplotypes. The method is also limited to analyzing two GV sites per assay.

PCT Publication No. WO 01/90418 submitted by Cai, et al., incorporated by reference herein in its entirety, depicts using a single molecule approach based on the simultaneous detection of two distinguishable luminescent labels that are specific to neighboring genetic markers, such as SNPs, from single chromosomes. However, this patent requires techniques for distinguishing luminescence, including color differentiation or luminescence lifetime, to determine haplotype. In contrast, the present invention can use a single label, or probe, to determine haplotype, and using certain protocols, it is preferable to use a single label or two probes labeled with the same indistinguishable dye.

Cai, et al., however, point out that by using single molecule detection and identification, the co-location of two markers on a given haploid can be rapidly determined. Traditionally, association studies have been successful only for simple, monogenic diseases involving a small number of markers, where the possible combinations of different haplotypes are limited. Therefore, the haplotypes can be deduced from genotypes by typing many individuals and by the availability of homozygotes and parental information. However, most diseases are complex and involve multiple genes. For polygenic association studies, many more markers are needed and, therefore, the number of possible haplotypes is large. In these cases, it is extremely difficult to infer the haplotype from the genotype. Many sophisticated algorithms have been developed for haplotype prediction and they are typically 70-90% accurate. Such accuracy is not useful when typing a large numbers of SNPs and also is not acceptable for clinical diagnostic purposes. In addition, it is often impossible or impractical to obtain parental genomic DNA. This raises a serious challenge: there is no easy way to directly determine a haplotype except when it is on the sex chromosomes where X and Y chromosome are sufficiently different to be distinguished in bulk methods.

As shown in Cai, et al., a genetic profile based on a genotype can be incomplete, because it fails to provide the locations of SNPs on two chromosomes. For example, consider two genetic markers (or SNPs), A and B, on the same gene. For a genotype of aA/bB (A and B presents the wild type or dominant genotype that naturally occurs; a and b represent two mutations.), there are two possible combinations of haplotypes, ab/AB and Ab/aB. The disease phenotype for the individual with ab/AB may be less severe compared to the individual with Ab/aB. This is because the individual with ab/AB has one intact copy of the gene, whereas the individual with Ab/aB has no intact copy on either chromosome. For cases like this, the ability to find out whether two mutations are on the same chromosome or on different chromosomes (haplotypes) in a routine clinical setting is particularly useful for future risk assessment and disease diagnostics.

Another conventional alternative for haplotyping is allele-specific polymerase chain reaction (allele-specific PCR (Ruano, G., et al., Haplotype of multiple polymorphisms resolved by enzymatic ampification of single DNA molecule, PNAS 87:6296-6300 (1990))), which is the most commonly used method for direct haplotyping. In these reactions, SNP-specific PCR primers are designed to distinguish and amplify a specific haplotype from two chromosomes. Such reactions require stringent reaction conditions and individual optimization for each target. Therefore, this approach is not suitable for a large scale and high throughput haplotyping. More importantly, such assays are subject to the length limitations of PCR amplification and are not capable of typing SNPs that are more than several kilobases (kb) apart. In addition, such an amplification-based typing is often complicated by the contamination of a small amount of genomic DNA other than the sample DNA during sample handling process.

Other haplotyping methods according to Cai, et al., include single sperm or single chromosome measurements (Ruano, supra; Zhang L., Whole genome amplification from a single cell: Implications for genetic analysis, PNAS 89:5847-5851 (1992); Vogelstein, B., Digital PCR, PNAS 96(16):9236-9241 (1999); Wahlestedt C., et al., Potent and nontoxic antisense oligonucleotides containing locked nucleic acids, PNAS 97:5633-5638 (2000)). In a single sperm sorting assay, PCR-amplified DNA from individual sorted sperm cells is genotyped. Multiple sperm cells (at least 3-5) from an individual are typed in order to have enough statistical confidence to reveal the two haplotypes. In principle, this sorting approach could be applied to chromosomes. However, this technique is complicated, and, so far, has been successful in only a few research labs. The molecular cloning method involves cloning a target region of an individual's DNA (or cDNA) into a vector, and genotyping the DNA obtained from single colonies. For each individual, multiple colonies are needed to obtain two haplotypes. This method has been used by many laboratories, but is very labor-intensive, time-consuming and can be difficult to perform in some cases. Researchers are forced to use it because there is no easy alternatives.

Finally, Cai, et al., point out that haplotyping by AFM (Atomic Force Microscopy) imaging (Wooley, A. T., et al., Direct haplotyping of kilobase-size DNA using carbon nanotubes probes, Nature Biotechnology 18:760-763 (2000); Taton, T. A., et al., Haplotyping by force, Nature Biotechnology 18:713-713 (2000)) is a newer approach to directly visualize the polymorphic sites on individual DNA molecules. This method utilizes AFM with high resolution single walled carbon nanotube probes to read directly multiple polymorphic sites in DNA fragments containing from 100-10,000 bases. This approach involves specific hybridization of labeled oligonucleotide probes to target sequences in DNA fragments followed by direct reading of the presence and spatial localization of the labels by AFM. However, the throughput and sensitivity of such systems remain to be demonstrated; currently 200 samples per day, each with 10 images, can be processed.

In summary, Cai, et al., point out that there is generally no easy way to determine a haplotype currently except by using the sex chromosomes. In contrast to Cai, et al., however, which requires using two distinguishable dyes to determine haplotype, a simpler and more effective approach has been developed in the present invention which can use a single dye or two indistinguishable dyes. In addition, unlike most prior art methods, which require DNA amplification by PCR or cloning and extensive optimization, the present invention establishes a more direct method for determining haplotype.

A simple and direct method for detecting haplotypes would be of significant value for diagnostics and biomedical research. This would be especially true for a method that could analyze multiple GV sites per assay, with GV sites spaced as much as 20 kilobases apart. An additional useful feature of a haplotype method would be the ability to determine GV haplotypes for multiple genetic regions in the same assay.

### BRIEF SUMMARY OF THE INVENTION

The present invention provides methods for directly identifying haplotypes of nucleic acids possessing sequence differences or specific polymorphic variants. The polymorphic variations may be insertions, deletions or single base replacements. Polymorphic sites analyzed may be greater than 20 kb apart. Methods of the present invention can be used to determine haplotypes for multiple GVs in multiple genetic regions in a single assay.

In general, the invention provides methods for enumerating (i.e., counting) nucleic acids that interact with at least one probe, at least two luminescent probes which are indistinguishable using certain techniques, or one luminescent and one nonluminescent probe where the probes interact with specific sequences on the target that represent sites of genetic variation. The probes interact sequentially or simultaneously with the target. Targets may include nucleic acids, nucleic acid fragments, plasmids and other molecules such as gene fragments and the like. The probes may be nucleic acids, oligonucleotides, nucleic acid variants such as PNAs or LNAs, peptides, proteins, dyes, lipids, drugs, or small molecules. Any combination of probe types may be used in a given experiment. Haplotype determination is based upon measurement of one or more parameters that are influenced by, or dependent upon, the probe(s).

In addition, the present invention combines the advantages of single-molecule detection of nucleotide markers with free-solution or sieved, single-molecule capillary electrophoresis techniques

Therefore, the present invention provides a method for determining genetic haplotype comprising (a) identifying a target nucleic acid molecule or gene fragment, said nucleic acid molecule or gene fragment comprising a haplotype of interest, by: (i) hybridizing a primer recognizing a first genetic variant, said variant correlating to a haplotype, to the target nucleic acid molecule or gene fragment and wherein a labeled primer dependent transcript is generated from the target nucleic acid or gene fragment; and (1) hybridizing at least one labeled probe, said probe recognizing a second genetic variant downstream from the primer, to the primer dependent transcript, or (2) hybridizing at least one unlabeled probe, said probe recognizing recognizing a second genetic variant downstream from the primer, to the primer dependent transcript; and (b) detecting at least one parameter displayed by one of the primer-dependent transcript, the at least one probe, or a primer-dependent transcript/probe complex, thereby correlating the displayed parameter to the haplotype.

Also provided is a method for determining genetic haplotype comprising (a) identifying a target nucleic acid molecule or gene fragment, said nucleic acid molecule or gene fragment comprising a haplotype of interest, by (i) hybridizing a primer recognizing a first genetic variant, said variant correlating to a haplotype, to the target nucleic acid molecule or gene fragment and wherein a primer dependent transcript is generated from the target nucleic acid or gene fragment; and (1) hybridizing at least one labeled probe, said probe recognizing a second genetic variant downstream from the primer, to the primer dependent transcript, or (2) hybridizing at least one unlabeled probe, said probe recognizing a second genetic variant downstream from the primer, to the labeled primer dependent transcript; and (b) detecting at least one parameter displayed by one of the labeled primer-dependent transcript, the at least one probe, or a primer-dependent transcript/probe complex, thereby correlating the displayed parameter to the haplotype.

Further provided is a method for determining genetic haplotype comprising (a) labeling a nucleic acid molecule or gene fragment with at least two probes, each probe recognizing a different genetic variation that defines a haplotype; and (b) detecting the nucleic acid molecule or gene fragment by measuring a sequential change in a single parameter displayed by the probes, thereby rendering the genetic haplotype determinable.

Additionally provided is a method for determining genetic haplotype comprising (a) labeling a nucleic acid molecule or gene fragment with at least two probes, each probe recognizing a different genetic variation that defines a haplotype; and (b) detecting the nucleic acid molecule or gene fragment by measuring a sequential change in at least two parameters displayed by the probes, thereby rendering the genetic haplotype determinable.

Moreover provided is a method for determining genetic haplotype comprising (a) labeling a nucleic acid molecule or gene fragment with at least two probes, each probe recognizing a different genetic variation that defines a haplotype; and (b) detecting the nucleic acid molecule or gene fragment by simultaneously measuring the parameters displayed by the probes, wherein the parameter is not cooperative hybridization, thereby rendering the genetic haplotype determinable.

Further provided is a method for determining genetic haplotype comprising (a) labeling a nucleic acid molecule or gene fragment with at least two probes, each probe recognizing a different genetic variation that defines a haplotype; and (b) detecting the nucleic acid molecule or gene fragment by simultaneously measuring at least two parameters displayed by the probes, wherein the parameter is not cooperative hybridization, and further wherein the probe is bound to a molecule selected from the group consisting of a microsphere, nanosphere and bar code particle, thereby rendering the genetic haplotype determinable.

Additionally provided is a method for determining genetic haplotype comprising (a) labeling a nucleic acid molecule or gene fragment with at least one probe, each probe recognizing a different genetic variation that defines a haplotype; and (b) detecting the velocity of the nucleic acid molecule or gene fragment by measuring the difference in time the probe displays a parameter measured by a first detector at a first position and a second detector at a second position, wherein the probe is bound to a molecule selected from the group consisting of a microsphere, nanosphere, bar code particle, and nanocrystal, thereby rendering the genetic haplotype determinable.

These and other features, aspects and advantages of the present invention will become better understood with reference to the following description, examples and appended claims.

### BRIEF DESCRIPTION OF THE SEVERAL VIEWS OF THE DRAWINGS

FIG. 1: An exemplary apparatus is depicted for rapid haplotyping by labeled single molecule fluorescence detection. Two excitation lasers 10 and 12 are focused through microscope objective 14 to excite DNA sample 24 which has been labeled with at least one probe. The emission of the probe in its excited state is collected by microscope objective 14, passes through polychroic beam splitter 13, and spectrally split with dichroic beam splitter 15 between two sensitive photon counting detectors 16 and 18. Detectors 16 and 18 are single photon counting avalanche photodiodes. Laser 10 or 12 can be operated at particular wavelengths depending upon the nature of the detection probe which will be excited upon contact with the laser beam. The detection channel from detector 16 is band pass filtered (filter not shown) to detect a predetermined wavelength emission. The detection channel from detector 18 is band pass filtered (filter not shown) to detect the same or a different predetermined wavelength emission. DNA labeled with two probes will be registered in both detectors. DNA labeled with one probe will be detected by a single detector 16 or 18. The intensity recorded by detectors 16 and 18 is cross-correlated to detect the presence of DNA fragments containing both labels. A pinhole 17 in the image plane of microscope objective 14 limits the field of view of two detectors 16 and 18 to the immediate vicinity of the overlapping, focused laser beams. A personal computer 22 houses a digital correlator card that computes the cross-correlation between the two detection channels in real-time.

FIG. 2: An exemplary apparatus is depicting a capillary flow cell 30. Laser beams 10 and 12 are optically focused on a narrow glass capillary tube that contains the liquid sample 24. An electric current is applied to the solution in the tube, causing fluorescent molecules to move through the tube in lockstep. As molecules pass through each laser beam 10 and 12, excitation of each fluorescent molecule takes place. Within a fraction of a second, the excited molecule relaxes, emitting a detectable burst of light. This light is detected by detectors 16 and 18. The excitation-emission cycle is repeated many times by each molecule in the length of time it takes for it to pass through the laser beam. The light bursts from a single fluorescent molecule are collected at right angles to the incident laser beam and focused by a microscope objective 14 onto light sensing detectors 16 and 18. A filter (not shown) is used to keep excitation light from the laser from reaching the detector. The time for passage of a fluorescent molecule between two laser beams is measured by PC 22.

### DETAILED DESCRIPTION OF THE INVENTION

Abbreviations and Definitions

Unless indicated otherwise, the terms defined below have the following meanings:

Haplotype: As used herein, the term “haplotype” refers to the set, made up of one allele of each gene, comprising the genotype. Also used to refer to the set of alleles on one chromosome or a part of a chromosome, i.e. one set of alleles of linked genes. In the context of the present invention a haplotype preferably refers to a combination of biallelic marker alleles found in a given individual and which may be associated with a phenotype.

Allele: As used herein, the term “allele” refers to any one of a series of two or more different genes that occupy the same position (locus) on a chromosome. Since autosomal chromosomes are paired, each autosomal locus is represented twice. If both chromosomes have the same allele, occupying the same locus, the condition is referred to as homozygous for this allele. If the alleles at the two loci are different, the individual or cell is referred to as heterozygous for both alleles.

Locus: As used herein, the term “locus” refers to the site in a linkage map or on a chromosome where the gene for a particular trait is located. Any one of the alleles of a gene may be present at this site.

Polymorphism: As used herein, the term “polymorphism” refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism, or SNP, is a single base pair change. Typically a single nucleotide polymorphism is the replacement of one nucleotide by another nucleotide at the polymorphic site. Deletion of a single nucleotide or insertion of a single nucleotide, also give rise to single nucleotide polymorphisms. In the context of the present invention “single nucleotide polymorphism” preferably refers to a single nucleotide substitution. Typically, between different genomes or between different individuals, the polymorphic site may be occupied by two different nucleotides.

Biallelic Marker: As used herein, the term “biallelic marker” refers to a polymorphism having two alleles at a fairly high frequency in the population, preferably a single nucleotide polymorphism.

Cross-correlation: Cross-correlation involves subjecting two raw data sets gⱼ and hₖ to analysis, whereby data sets from each detector (preferably photon detectors) are subjected to the following formula:

$${Corr ( g , h )}_{j} ≡ {\sum }_{k = 0}^{N - 1} {g}_{j + k} {h}_{k} , \\ for j = - ( N - 1 ) , - ( N - 2 ) , … , - 1 , 0 , 1 , … , N - 1$$

where N is the total number of data points. The data cross-correlations will be large at values of j where the first data set from a detector [preferably photon counts above a background level] (g) resembles the data set (h) from a second detector [preferably above a background level] at some lag time (j) that corresponds to the time for specific molecules to pass from the first detector to the second detector [preferably in a single molecule analytical system]. In a single molecule electrophoresis instrument with an electric field applied to the sample, the lag time (j) for detection between photon detectors arrayed along the length of capillary is related to the electrophoretic velocity of a detected molecule. In the same instrument with no electric field supplied to the capillary, but with sample pumped,through the capillary, the lag time (j) for photon burst detection is the same for all molecules and is related to the pumping speed.

Dye: As used herein, the term “dye” refers to a substance used to color materials or to enable generation of luminescent or fluorescent light. A dye may absorb light or emit light at specific wavelengths. A dye may be intercalating, noncovalently bound or covalently bound to probe and/or target. Dyes themselves may constitute probes as in probes that detect minor groove structures, cruciforms, loops or other conformational elements of nucleic acids. Dyes may include BODIPY and ALEXA dyes, Cy[n] dyes, SYBR dyes, ethidium bromide and related dyes, acridine orange, dimeric cyanine dyes such as TOTO, YOYO, BOBO, TOPRO POPRO, and POPO and their derivatives, bis-benzimide, OliGreen, PicoGreen and related dyes, cyanine dyes, fluorescein, LDS 751, DAPI, AMCA, Cascade Blue, CL-NERF, Dansyl, Dialkylaminocoumarin, 4′,5′-Dichloro-2′,7′-dimethoxyfluorescein, 2′,7′-Dichlorofluorescein, DM-NERF, Eosin, Erythrosin, Fluoroscein, Hydroxycourmarin, Isosulfan blue, Lissamine rhodamine B, Malachite green, Methoxycoumarin, Naphthofluorescein, NBD, Oregon Green, PyMPO, Pyrene, Rhodamine, Rhodol Green, 2′,4′,5′,7′-Tetrabromosulfonefluorescein, Tetramethylrhodamine, Texas Red, X-rhodamin and other dyes that interact with or may be conjugated to probes or targets. Those skilled in the art will recognize other dyes which may be used within the scope of the invention. This is not an exclusive list and includes all dyes now known or known in the future which could be used to allow detection of the labeled nucleotides of the invention.

Probe: As used herein, the term “probe” refers to a defined nucleic acid segment, or a biochemical or biological molecule or complex that can be used to identify a specific nucleotide sequence present in targets. The defined nucleic acid segment comprises a nucleotide sequence complementary to the specific nucleotide sequence to be identified.

Fluorescence Lifetime: As used herein, the term “fluorescence lifetime” refers to the time required by a population of N excited fluorophores to decrease exponentially to N/e by losing excitation energy through fluorescence and other deactivation pathways.

Fluorescence Polarization: As used herein, the term “fluorescence polarization” refers to the property of fluorescent molecules in solution, excited with plane-polarized light, to emit light back into a fixed plane (i.e. the light remains polarized) if the molecules remain stationary during the excitation of the fluorophore.

Mass: As used herein, the term “mass” refers to a physical “constant of proportionality” relating force and acceleration

Net Charge: As used herein, the term “net charge” refers to the arithmetic sum, taking polarity into account, of the charges of all the atoms taken together for a molecule.

Shape: As used herein, the term “shape” refers to the three-dimensional structure of a molecule or molecular complex and the variations of a such three-dimensional structure when a molecule or molecular complex is in solution.

Diffusion: As used herein, the term “diffusion” refers to the slow motion of molecules from one place to another.

Electrophoretic Velocity: As used herein, the term “electrophoretic velocity” refers to the velocity of a charged or uncharged analyte under the influence of an electric field relative to the background electrolyte. Electrophoretic velocity in a capillary system may be a composite measure of electrokinetic velocity and electroosmotic force.

Fluorescence: As used herein, the term “fluorescence” refers to the emission of radiation, generally light, from a material during illumination by radiation of usually higher frequency or from the impact of electrons.

Fluorescence Intensity: As used herein, the term “fluorescence intensity” refers to the output of a detection system that measures the radiation from a fluorescing sample. It also refers to the number of photons detected per unit time (preferably milliseconds and preferably above a background threshold).

Luminescence: As used herein, the term “luminescence” refers to the emission of light by a substance for any reason other than a rise in temperature.

Luminescence Intensity: As used herein, the term “luminescence intensity” refers to the output of a detection system that measures the light emission from a luminescent sample. It also refers to the number of light emissions detected per unit time (preferably milliseconds and preferably above a background threshold).

Chemiluminescence: As used herein, the term “chemiluminescence” refers to luminescence produced by the direct transformation of chemical energy into light energy. Also called chemoluminescence.

Chemiluminescence Intensity: As used herein, the term “chemiluminescence intensity” refers to the output of a detection system that measures the light emission from a chemiluminescent sample. It also refers to the number of photons detected per unit time (preferably milliseconds and preferably above a background threshold).

Light Absorption: As used herein, the term “light absorption” refers to the light energy (wavelengths) not reflected by an object or substance.

Electrical Reactance: As used herein, the term “electrical reactance” refers to opposition offered to the flow of AC by the inductance or capacity of a part

PNA: As used herein, the term “PNA” refers to Peptide Nucleic Acid (PNA) oligomers—a new class of molecules: analogs (mimics)of DNA in which the phosphate backbone is replaced with an uncharged “peptide-like” (polyamide) backbone.

LNA: As used herein, the term “LNA” refers to Locked Nucleic Acid which is a novel class of nucleic acid analogs. LNA monomers are bicyclic compounds structurally similar to RNA nucleosides comprising a furanose ring conformation restricted by a methylene linker that connects the 2′-O position to the 4′-C position. For convenience, all nucleic acids containing one or more LNA modifications are called LNA. LNA oligomers obey Watson-Crick base pairing rules and hybridize to complementary oligonucleotides. The design, synthesis and hybridization of LNA probes are well known in the art.

DNX: As used herein, the term “DNX” refers to nucleic acid probes that are composed of one or more crosslinking nucleotide analogs. The analogs promote covalent bonding between the probe and target nucleic acid upon hybridization, and may require photoactivation for crosslinking to occur.

Phenotype: As used herein, the term “phenotype” refers to any visible, detectable or otherwise measurable property of an organism such as symptoms of, or susceptibility to a disease.

Hybridization: As used herein, “hybridization” refers to the formation of a duplex structure by two single stranded nucleic acids due to complementary base pairing. Hybridization can occur between exactly complementary nucleic acid strands or between nucleic acid strands that contain minor regions of mismatch. Specific probes can be designed that hybridize to one form of a biallelic marker and not to the other and therefore are able to discriminate between different allelic forms. Allele-specific probes are often used in pairs, one member of a pair showing perfect match to a target sequence containing the original allele and the other showing a perfect match to the target sequence containing the alternative allele. Hybridization conditions should be sufficiently stringent that there is a significant difference in hybridization intensity between alleles, and preferably an essentially binary response, whereby a probe hybridizes to only one of the alleles. Stringent, sequence specific hybridization conditions, under which a probe will hybridize only to the exactly complementary target sequence are well known in the art (Sambrook et al., Molecular Cloning—A Laboratory Manual, Third Edition, Cold Spring Harbor Press, N.Y., 2001). Stringent conditions are sequence dependent and will be different in different circumstances. Generally, stringent conditions are selected to be about 5° C. lower than the thermal melting point (Tm) for the specific sequence at a defined ionic strength and pH.

Application of Multiple Probe Haplotype Determination

Diploid cells display two haplotypes at any gene or other chromosomal segment having at least one distinguishing variance. Haplotype variations are correlated more strongly with phenotype than many well-studied single-nucleotide variances, e.g., single-nucleotide polymorphisms. Therefore, studying haplotypes is valuable for understanding the genetic basis of a variety of phenotypes including disease predisposition or susceptibility, response to therapeutic interventions and other phenotypes of interest in medicine.

The first generation of markers were RFLPs, which are variations that modify the length of a restriction fragment. But methods used to identify and to type RFLPs are relatively material- and time-intensive. The second generation of genetic markers were VNTRs which can be categorized as either minisatellites or microsatellites. Minisatellites are tandemly repeated DNA sequences present in units of 5-50 repeats which are distributed along regions of the human chromosomes ranging from 0.1 to 20 kilobases in length. Since they present many possible alleles, their informative content is very high. Minisatellites are scored by performing Southern blots to identify the number of tandem repeats present in a nucleic acid sample from the individual being tested. However, there are only 10⁴ potential VNTRs that can be typed by Southern blotting. Moreover, both RFLP and VNTR markers are costly and time-consuming to develop and assay in large numbers.

GVs, such as SNPs or biallelic markers, can be used in the same manner as RFLPs and VNTRs but offer several advantages. SNPs are densely spaced in the human genome and represent the most frequent type of variation. An estimated number of more than 10⁷ sites are scattered along the 3×10⁹ base pairs of the human genome. Therefore, SNPs occur at a greater frequency and with greater uniformity than RFLP or VNTR markers which means that there is a greater probability that such a marker will be found in close proximity to a genetic locus of interest. SNPs are less variable than VNTR markers but are mutationally more stable.

Additionally, the different forms of a characterized SNP are often easier to distinguish and can therefore be typed easily on a routine basis. Biallelic markers have single nucleotide based alleles and they have only two common alleles, which allows highly parallel detection and automated scoring. Thus, the methods of the present invention offer the possibility of rapid, high-throughput haplotyping of a large number of individuals.

Biallelic markers are densely spaced in the genome, sufficiently informative and can be assayed in large numbers. The combined effects of these advantages make biallelic markers extremely valuable in genetic studies. Biallelic markers can be used in linkage studies in families, in allele sharing methods, in linkage disequilibrium studies in populations, in association studies of case-control populations. Biallelic markers allow association studies to be performed to identify genes involved in complex traits. Association studies examine the frequency of marker alleles in unrelated case and control populations and are generally employed in the detection of polygenic or sporadic traits. Association studies may be conducted within the general population and are not limited to studies performed on related individuals in affected families (linkage studies). Biallelic markers in different genes can be screened in parallel for direct association with disease or response to a treatment. This multiple gene approach is a powerful tool for a variety of human genetic studies as it provides the necessary statistical power to examine the synergistic effect of multiple genetic factors on a particular phenotype, drug response, sporadic trait, or disease state with a complex genetic etiology.

In one aspect of the invention for haplotype determination, target genomic DNA is cut into fragments using one or more restriction endonucleases. Any source of nucleic acids, in purified or non-purified form, can be utilized as the starting nucleic acid, provided it contains or is suspected of containing the specific nucleic acid sequence desired. DNA or RNA may be extracted from cells, tissues, body fluids and the like as described below. While nucleic acids for use in the genotyping methods of the invention can be derived from any mammalian source, the test subjects and individuals from which nucleic acid samples are preferably human.

As for the source of the genomic DNA to be subjected to analysis, any sample from a living being can be used without any particular limitation. These samples include biological samples which can be tested by the methods of the present invention described herein and include human and animal body fluids such as whole blood, serum, plasma, cerebrospinal fluid, urine, lymph fluids, and various external secretions of the respiratory, intestinal and genitourinary tracts, tears, saliva, milk, white blood cells, myelomas and the like; biological fluids such as cell culture supernatants; fixed tissue specimens including tumor and non-tumor tissue and lymph node tissues; and bone marrow aspirates and fixed cell specimens. The preferred source of genomic DNA used in the present invention is from peripheral venous blood of each donor. Techniques to prepare genomic DNA from biological samples are well known to those skilled in the art.

For example, DNA samples may be prepared from peripheral venous blood as follows: Thirty ml of peripheral venous blood can be taken from a donor in the presence of EDTA. Cells (pelleted) may be collected after centrifugation for 10 minutes at 2000 rpm. Red cells may be lysed in a lysis solution (50 ml final volume: 10 mM Tris pH 7.6; 5 mM MgCl₂; 10 mM NaCl). The solution is then centrifuged (10 minutes, 2000 rpm) as many times as necessary to eliminate the residual red cells present in the supernatant, after resuspension of the pellet in the lysis solution. The pellet of white cells is then lysed overnight at 42°C. with 3.7 ml of lysis solution composed of (a) 3 ml TE 10-2 (Tris-HCl 10 mM, EDTA 2 mM)/NaCl 0.4 M; (b) 200 μl SDS 10%; and (c) 500 μl proteinase K (2 mg proteinase K in TE 10-2/NaCl 0.4 M).

The two strands of the target nucleic acid derived from the venous blood serum above, or from any source of genomic DNA, are digested into fragments by endonucleases and are dissociated. Depending on the protocol described below, either one or two probe peptide nucleic acids (PNAs) (U.S. Pat. No. 5,539,082 to Nielsen, incorporated by reference herein in its entirety) specific to a singular or different GV site, may be hybridized to the nucleic acid targets. The hybridized mixture of target nucleic acids and GV probes is analyzed to detect the simultaneous binding of the one or two GV probes to individual target nucleic acid fragments.

Any GV markers known in the art may be used with the target genomic DNA and mRNA of the present invention in the haplotyping methods described herein, for example in anyone of the following web sites in Table 1:

TABLE 1
The Genetic Annotation Initiative (http://Ipg.nci.nih.gov/Ipg_small). An NIH
run site which contains information on candidate SNPs thought to be related
to cancer and tumorigenesis generally.
dbSNP Polymorphism Repository (http://www.ncbi.nlm.nih.gov/SNP/). A
more comprehensive NIH-run database containing information on SNPs with
broad applicability in biomedical research.
HUGO Mutation Database Initiative
(http://ariel.its.unimelb.edu.au/˜cotton/mdi.htm). A database meant to
provide systematic access to information about human mutations including
SNPs. This site is maintained by the Human Genome Organization (HUGO).
Human SNP Database (http://www-genome.wi.mit.edu/snp/human/index.html).
Managed by the Whitehead Institute for Biomedical Research Genome Institute,
this site contains information about SNPs resulting from the many Whitehead
research projects on mapping and sequencing.
Japanese SNPs in the Human-Genome SNP database (http://snp.ims.u-tokyo.ac.jp/).
This website provides access to SNPs that have been organized by chromosomes
The site is run by the University of Tokyo.
HGBase (http://hgbase.interactiva.de/). HGBASE is an attempt to
summarize all known sequence variations in the human genome, to facilitate
research into how genotypes affect common diseases, drug responses, and
other complex phenotypes, and is run by the Karolinska Institute of Sweden.
The SNP Consortium Database (http://snp.cshl.org/). A collection of SNPs
and related information resulting from the collaborative effort of a number of
large pharmaceutical and information processing companies.
GeneSNPs (http://www.genome.utah.edu/genesnps/). Run by the University
of Utah, this site contains information about SNPs resulting from the U.S.
National Institute of Environmental Health's initiative to understand the
relationship between genetic variation and response to environmental stimuli
and xenobiotics.

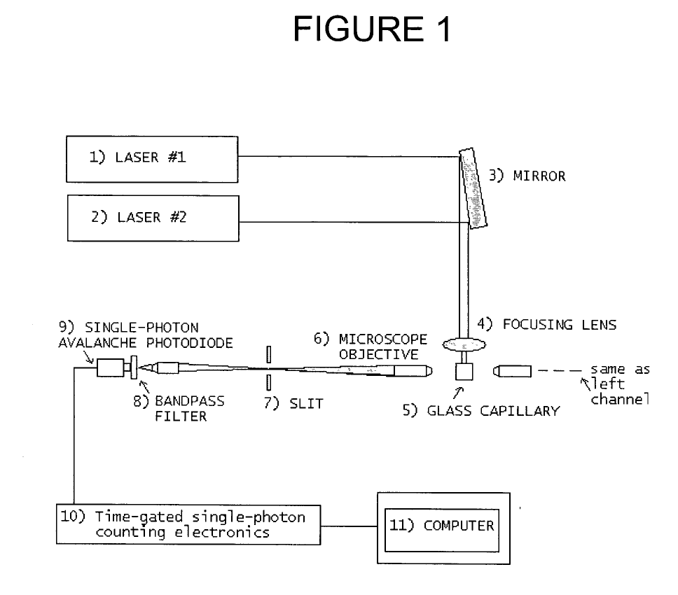

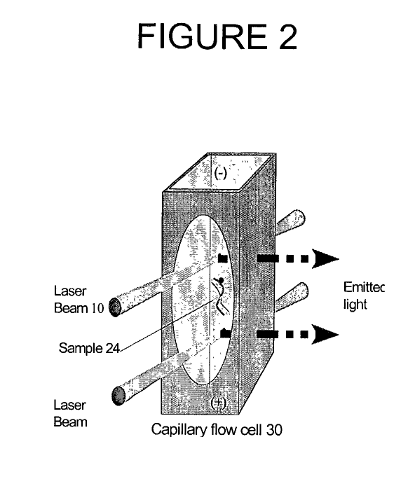

Coincident hybridization of the GV probes is detected via single-molecule electrophoresis (Castro, A., et al., Single-molecule electrophoresis, Anal. Chem. 67(18):3181-86 (1995)). The single molecule electrophoresis instrument depicted in FIGS. 1 and 2 provides an ultrasensitive means to detect individual fluorescently tagged molecules.

Laser epiillumination is used in combination with confocal fluorescence detection to probe an extremely small volume of the solvent. Two excitation lasers 10 and 12 are focused through microscope objective 14 to excite DNA sample 24 which has been labeled with at least one probe. Using a dilute DNA solution, labeled DNA fragments will not reside within the focused laser beams for a period of time. When an individual DNA diffuses into the excitation region, the label or labels on the DNA will become detectable. The fluorescence is collected by microscope objective 14, passes through polychroic beam splitter 13, and spectrally split with dichroic beam splitter 15 between two sensitive photon counting detectors 16 and 18.

The exemplary apparatus is based on a known laser epi-illuminated and confocal fluorescence emission collection design depicted in Cai, et al., supra. The linear dimensions of the probe volume for the sample 24 are on the order of a micron or less resulting in a probe volume on the order of 1 femtoliter (fl) (Rigler, et al. 1993). Laser 10 or 12 can be operated at particular wavelengths depending upon the nature of the detection probe which will be excited upon contact with the laser beam. For example, Laser 10 may be an Ar⁺ laser operating at 496 nm to excite a fluorescein fluorophore. Laser 12 may be a helium neon laser operating at 633 nm to excite the fluorophore N,N′biscarboxypentyl-5,5′-disulfonatoindodicarbocyanine (Cy5).

Detectors 16 and 18 are single photon counting avalanche photodiodes. The detection channel from detector 16 is band pass filtered (filter not shown) to detect, e.g., fluorescein emission. The detection channel from detector 18 is band pass filtered (filters not shown) to detect, e.g., Cy5 emission. A pinhole 17 in the image plane of microscope objective 14 limits the field of view of two detectors 16 and 18 to the immediate vicinity of the overlapping, focused laser beams.

In a first embodiment, a laser beam is optically focused on a narrow glass capillary tube that contains the liquid sample (FIG. 2). An electric current is applied to the solution in the tube, causing fluorescent molecules to move through the tube in lockstep. As molecules pass through the laser beam, excitation of each fluorescent molecule takes place. Within a fraction of a second, the excited molecule relaxes, emitting a detectable burst of light. This excitation-emission cycle is repeated many times by each molecule in the length of time it takes for it to pass through the laser beam. The light bursts from a single fluorescent molecule are collected at right angles to the incident laser beam and focused by a microscope objective onto a light sensing detector. A filter is used to keep excitation light from the laser from reaching the detector. The time for passage of a fluorescent molecule between two laser beams is measured. This characteristic electrophoretic velocity is dependent upon the size, charge and shape of each molecule. Electrophoretic velocity is one of the parameters used to differentiate probe and target molecules in specific embodiments of the haplotyping technology described. The instrument detects hundreds of molecules per second.

In a second embodiment, sample 24, a microliter drop (e.g., 5 microliters) of a dilute solution of labeled DNA in this exemplary apparatus, may be suspended on the underside of a microscope coverslip. The coverslip is mounted on a scanning stage to allow the fluorescence detection probe volume to be raster scanned through the volume of the sample droplet. A personal computer 22 houses a commerically available digital correlator card (ALV 5000/E) that computes the cross-correlation between the two detection channels in real-time.

When an individual DNA fragment in sample 24 diffuses into the excitation region defined by microscope objective 14, the fluorescently-labeled probes on the DNA fragment will fluoresce. The fluorescence is collected and spectrally split between two sensitive detectors 16 and 18. Signals from DNA fragments that contain two probes will be registered in both detectors. A signal from a DNA fragment with only one hybridization probe will be registered by only a single detector. The intensity recorded by each detector is cross-correlated by computer 22 to look for instances where one or two probes are present on the same DNA fragment.

Single-Molecule Electrophoresis

The single-molecule electrophoresis technique consists of measuring the electrophoretic velocity of individual molecules-the velocity at which molecules move in solution under the influence of an electric field-and identifies them by comparing their measured velocity with the velocity characteristic of a particular molecular species. The electrophoretic velocity of a molecule is determined by its size, shape, and ionic charge and by the chemical environment of the solution in which it is contained. The electrophoretic velocity therefore provides a unique identification signature of each molecular species.

The apparatus for single-molecule electrophoresis consists of a laser source split into two beams, a sample compartment, light-collection optics, two single photon detectors, and detection electronics under computer control. The sample compartment contains two reservoirs, one of which contains a cathode and the other, an anode. The reservoirs hold the solution that is being analyzed and are connected by tubing to a the capillary cell. The two laser beams, which are focused at the capillary cell, produce two 5-micron spots separated by a distance of 250 microns.

When a voltage is applied to the electrodes, the molecules in the solution migrate toward the cathode or anode, depending on their charge. As the individual molecules in the solution pass through the two laser-illuminated spots, they emit bursts of fluorescence. The photons from each burst are then collected by a microscope objective and detected by a single-photon avalanche photodiode. The detection electronics reject Raman and Rayleigh scattering by the use of a time-gated window set to detect only delayed fluorescence photons. The instrument measures the time it takes for each molecule to travel the distance between the two laser beams and then uses this information to calculate the electrophoretic velocity of the molecule. The computer then produces a histogram of electrophoretic velocities which show a peak for every chemical species present in the sample.

Although the single-molecule electrophoresis technique relies on measuring molecular fluorescence, non-fluorescent molecules may be detected by attaching a fluorescent tagging molecule to them. In addition, some of the experimental conditions such as buffer composition, pH, viscosity, inner-surface capillary coating, excitation and emission wavelengths, among others, can be optimized to achieve the best separation of the particular sample components being analyzed. In fact, many of the analytical protocols specially developed for capillary electrophoresis separations are directly applicable to the present technique. For many years, researchers have optimized various capillary electrophoresis methods for the separation of a large variety of chemical species ranging from small organic and inorganic ions, to various kinds of pharmaceutical drugs and natural products.

The new method described here promises to combine the advantages of free-solution capillary electrophoresis (system automation, speed, and reproducibility) with the unsurpassed sensitivity of single-molecule detection. The sensitivity and versatility of the method may open the way to develop fluorescence immunoassay, hybridization, and DNA fingerprinting techniques without the need for extensive DNA amplification using the polymerase chain reaction (PCR) or other methods. Although PCR is a highly effective amplification mechanism, the use of many PCR cycles may introduce ambiguities arising from contamination and by mechanisms not yet fully understood. Besides the demonstrated ability for the analysis of single fluorophores, mixtures of nucleic acids and of proteins, the technique may find applications in many other fields that require the ultra-sensitive analysis of sample components.

Sample prepared as above is pumped into a square, glass capillary tube (200 μm on a side). A circular laser beam, 5 μm in diameter, passes perpendicularly through the loaded capillary. Laser-induced fluorescence is detected using suitably sensitive detectors (single-photon avalanche photodiodes, or “SPADs”) positioned at right angles to the incoming laser beam. The interrogation volume of the system is determined by the diameter of the laser beam and by the segment of the laser beam selected by the optics that directs light to the detectors. In this example, the interrogation volume is set such that, with an appropriate sample concentration, single molecules (single nucleic acid target fragments) are present in the interrogation volume during each time interval over which observations are made.

Two detectors are used. The optical path for each detector is trained on the same region of the laser beam and, therefore, each detector “interrogates” the identical volume. Two different peptide nucleic acid GV probes are used, each of which hybridizes to a different GV on the same DNA strand. One probe is end-labeled with fluorescent Rhodamine-6G while the second probe is end-labeled with fluorescent BODIPY-TR. The probes are excited at 532 nm, but each probe emits fluorescence at a different, discernable wavelength. The optical path to each detector incorporates light filters such that each SPAD will detect only one of the two fluorescent GV probes used in the experiment. A potential of 2000 Volts is passed through the sample to move sample components through the capillary. Data is collected on the number of fluorescent photons observed at each detector in successive 2 ms intervals. Collected data is analyzed to determine when fluorescence was detected simultaneously at both wavelengths. Coincident detection of both GV probes indicates that both GV probes have hybridized to a single nucleic acid fragment. At probe concentrations below 1 pM coincident detection of fluorescent probes does not occur. Consequently a homogeneous assay format can be used and unbound probes need not be removed prior to assay of the sample. This is similar to the flow cytometry method demonstrated by Castro, A., et al., (Single-molecule detection of specific nucleic acid sequences in unamplified genomic DNA, Anal. Chem. 69(19):3915-20 (1997)) for detecting DNA. When genomic DNA is the target, hybridization of the two PNA probes indicates the haplotype for the GV probes on individual DNA fragments.

Alternatives to fluorescence can be used to detect coincident or sequential probe interaction with targets. Detectable parameters can include—mass, charge, shape, fluorescence lifetime, fluorescence polarization, diffusion, and the like. Probes may be nucleic acids, oligonucleotides, PNAs, LNAs, peptides, proteins or any other molecule that can interact specifically with a GV site (See, e.g., U.S. Pat. No. 5,539,082 to Nielsen, incorporated by reference herein in its entirety; See also http://www.exiquon.com, last visited Oct. 24, 2002). Probes may affect a single parameter, or multiple parameters can be analyzed, with each parameter affected by one or more probes.

Coincident Two Probe Haplotyping

In another aspect of the invention, one fluorescent GV probe and one mass GV probe (non-fluorescent) are used. The mass probe consists of single nanospheres covalently bound to single peptide nucleic acid (PNA) 15-mers. The nanospheres used are synthesized and purified to generate a nanosphere population with a precise molecular weight and charge (Bhalgat, M. K., et al., Green- and red-fluorescent nanospheres for the detection of cell surface receptors by flow cytometry, J. Immunol. Methods 219(1-2):57-68 (1998)). Consequently, the nanosphere-PNA probe also has a precise molecular weight and charge. An electric current is applied to the sample solution in the capillary and molecules in the solution move through the capillary with a rate dependent upon the charge/mass ratio of each molecule. A second laser beam and associated light detector are trained on the capillary downstream of the first laser beam/detector. The second laser beam is configured such that a molecule that passes through the first beam will pass through the second beam as well. Both beam/detector systems measure fluorescence from the first GV probe.

Custom software is used to measure the time for passage of a fluorescent molecule between the first and second detectors. This transit time (electrophoretic velocity) is dependent upon the charge/mass ratio of the observed molecule or complex. Three types of fluorescent molecules/complexes are observable in this system:

(a) fluorescent probe;

(b) fluorescent probe+target; and

(c) fluorescent probe+mass probe+target.

Each of the three types of molecules has a specific transit time in solution and can be distinguished (Long, D., et al., Electrophoretic mobility of composite objects in free solution: application to DNA separation, Electrophoresis 17(6):1161-6 (1996)). In this example, coincidence is detected by measuring fluorescence of the probe-target complex and the change in electrophoretic velocity (altered charge/mass ratio) created by binding the mass probe.

Sequential Two Probe Haplotyping

Sequential interaction of probes with target nucleic acids also can be used to determine haplotypes. The sequential interaction of probes with target nucleic acids can be detected at a single detector over a time interval, or at separate detectors. As long as a means of distinguishing a specific target molecule is maintained, sequential interaction of probes can be detected. Detection of sequential probe binding is particularly useful under conditions where one or more of the probes does not bind tightly to the target nucleic acids.

An example of monitoring sequential probe interaction with target nucleic acids is presented. The probes in this example consist of a mass probe (PNA plus nanosphere) and a fluorescent oligonucleotide probe. The target nucleic acid fragments also are fluorescent in this example. The target and second probe emit fluorescent light of different, discernable wavelengths. Individual fluorescent target nucleic acid fragments are tracked as they move through a glass capillary in response to an electric field. A laser beam is configured to generate laser-induced fluorescence along much of the capillary length and a CCD detector is used to detect fluorescence. Fluorescent labeled nucleic acid fragments moving through the capillary are observed to move from pixel to pixel on the CCD detector (Shortreed, M. R., et al., High-throughput single-molecule DNA screening based on electrophoresis, Anal. Chem. 72(13):2879-85 (2000)). Nucleic acids have a uniform charge/mass, and consequently all fragments will move at a specific velocity in the system. Nucleic acid fragments that bind the mass probe have a new, specific velocity (due to a change in charge/mass ratio). Binding of the second probe is detected when the nucleic acid target becomes fluorescent at the wavelength associated with the second probe.

Multiplex analysis of several GV sites in a genetic region can be accomplished in a single assay using the present invention by using different, discernable features for each GV site. For example, four probes—two with discernable fluorescence and two mass probes (each of a different, discernable mass) can be used with a fluorescent target that is discernable from the fluorescent probes. Such a system can be used to determine the haplotype at four distinct GV sites.

Single-particle electrophoresis of target and probes (Castro, et al., Anal. Chem. 67, supra) can be used with the invention to analyze multiple genetic regions in a single assay. Each genetic region can be analyzed for multiple GV sites. In this application of the invention target fragment sizes are established such that each target genetic region (nucleic acid fragment) is discernable electrophoretically from other target genetic regions to be assayed. Likewise, probe masses and charges are established such that the various combinations of probes and target for each analyzed genetic region are discernable electrophoretically. Consequently, charge/mass ratio is used to identify the genetic regions, and probe interaction with the target is detected via altered charge/mass ratio or another probe parameter (fluorescence, etc.)

### EXAMPLES

The following experimental examples are offered by way of illustration and not by way of limitation.

### Example 1

Haplotype Single Probe Detection

In one aspect of the invention for haplotype determination a first GV site is detected by a specific oligonucleotide primer. Transcription is initiated from the primer using thermal DNA polymerase, and extension products are generated. Thermal cycling is instituted for 30 cycles to generate multiple extension products from each template. A single fluorescent PNA probe (labeled with Alexa 680 dye) that hybridized to a downstream GV on the extension product is added to the extension products under conditions that enable hybridization of the PNA to the extension product. After a 30 minute incubation at room temperature unhybridized PNA is removed from the sample by centrifugation of the sample through a Microcon 30YM filter/concentrator. Sample material retained by the filter is resuspended in 30 mM gly-gly buffer, pH 8.2. If the second GV site is present on the extension product, sample retained on the Microcon filter will include extension product-PNA hybrids. The sample is diluted to a final estimated concentration of 10-500 fM of extension product. Samples are then analyzed using a single-molecule electrophoresis instrument. Excitation of the sample is accomplished using a solid state laser at 1.5 mW power output per laser beam and 635 nm excitation wavelength. Two laser beams and two detectors with filters appropriate to detect fluorescent emission from the Alexa 680 dye are used as described earlier to determine the electrophoretic velocity of molecules in the sample. Fluorescent molecules with a velocity other than that of free PNA are enumerated. Such molecules are indicative of extension product-PNA hybrids and serve to indicate the haplotype.

Alternatively, sample prepared as above can be analyzed via the single-molecule electrophoresis instrument, but under conditions whereby sample is pumped through the detection capillary and no electric field is present. In this instrument configuration all molecules pass by the detector(s) at the same velocity. A control sample (PNA probe plus non-target DNA), processed to remove unbound PNA in a manner identical to that of a sample, is analyzed for fluorescence molecules (detected by photon bursts) and fluorescence is compared between the control and sample. Few fluorescent molecules will be detected in the control sample and numerous fluorescent molecules will be detected in the sample if both GVs are present and hybiridization of the probe has occurred.

In another aspect of the invention for haplotype determination a first GV site is detected by a specific oligonucleotide primer. An unlabeled PNA probe, specific for the second GV is added to the sample and transcription is initiated from the primer using thermal polymerase. Aminoallyl dUTP is incorporated into the extension product via transcription. If the second GV is present downstream of the primer, hybridized PNA probe will block transcription at that point. Thirty cycles of thermal cycling are used to generate multiple extension products from the template. The amine-reactive Alexa 680 dye is conjugated to the amines incorporated into the extension products as described in the ARES labeling kit. Unincorporated dye is removed from the sample by centrifugation of the sample through a Microcon 30YM filter/concentrator. Sample retained on the filter is resuspended in 30 mM gly-gly buffer at pH 8.2. The sample is diluted to a final estimated concentration of 10-500 fM of extension products. Samples are then analyzed using a single-molecule electrophoresis instrument. Excitation of the sample is accomplished using a solid state laser at 1.5 mW power output per laser beam and 635 nm excitation wavelength. Two laser beams and two detectors with filters appropriate to detect fluorescent emission from the Alexa 680 dye are used as described earlier to determine the electrophoretic velocity of molecules in the sample. If the second downstream GV site is present in the sample, then the PNA will block transcription at that site and transcription products of a fixed length will be generated. Fluorescence from these products will be detected at a single velocity using a single-molecule electrophoresis instrument. If the GV site is not present downstream then various lengths of transcription products will be generated. Fluorescence from these extension products will not be detected at a single velocity when analyzed using a single-molecule electrophoresis instrument.

### Example 2

Coincident Hybridization

Rationale: Use Alexa 680 labeled target and probe to detect coincident hybridization of two nucleic acid probes to target nucleic acid. Coincident hybridization will be deemed to have occurred at the molecular level when detected fluorescence intensity of molecules (and molecular hybridization complexes) analyzed using a single molecule detection instrument exceeds that of target, probes, and single-probe-target hybridization complexes. SME analysis of single stranded M13mp18 (ssM13) labeled with Alexa 680 yields photon bursts of 20 to 90 photons per 2 msec “bin”, with a rare events over 90 photons. Three double strand fragments generated by a Nci I restriction digest of M13mp18 RF (dsM13) labeled with Alexa 680 generate photon bursts in the 20 to 80 photon per 2 msec bin, with very rare events over 80 photons. The two samples were combined with the ssM13 Alexa680 (target) and the dsM13 Alexa 680 restriction fragments (probes). Following denaturation and renaturation one strand from each restriction fragment can hybridize in a non-overlapping manner to the ssM13 target. The number of photons produced by these hybrid molecules will be the sum of the photons given off by the ssM13 target (max of ˜90), and the photons given off by each single-strand probe that hybridizes (max of ˜40 photons for each probe). Hybrids will yield events with a higher average number of photon emissions compared with either the single strand target alone, or the double strand probes alone. This experimental design also allows for a control where both the labeled target and the labeled double strand probe are combined but not denatured. This control sample contains the same concentration of molecules as the experimental sample but should not yield events with more than a max of ˜90 photons per 2 msec since no single-strand probes are available to form hybrids with the single-strand target.

Methods: Restriction Digest to Generate Probe Fragments. 1 μg of M13mp18 RF (NEB) was digested with Nci I to yield three fragments of approximately 4 kb, 2 kb, and 0.5 kb. The restriction digest was phenol-extracted and ethanol-precipitated. The pellet was resuspended in 40 μl of TE buffer, pH 8.0.

1. Fluorescent Labeling of DNA Target and Probes. Functional amine groups were added to 1 μg of untreated ssM13mp18 (NEB) and 1 μg of Nci I digested DNA using the Label IT Amine Modifying Kit (Mirus, Madison, Wis.). DNA was incubated for one hour with the amine modifying reagent at 37° C. Each sample was then ethanol precipitated and resuspended in 5 μl of water. The Alexa 680 succinimidyl ester dye was coupled to the amine modified DNA using the coupling protocol provided in the ARES Alexa 680 kit (Molecular Probes, Eugene, Oreg.). (Incubate 1-5 μg of DNA in 5 μl of H₂O with 3 μl 25 mg/ml sodium bicarbonate, 2 μl of dye reagent. Each reaction was then ethanol precipitated and applied to a Microcon-30 column (Millipore). 1 ml of water was passed over each Microcon-30 to rid the sample of any trace amounts of unreacted amine-modifying reagent or Alexa dye. The final sample volume was 0.2 ml.

2. Hybridization Reactions and Single Molecule Analysis. Two samples were prepared by combining Alexa 680-labeled single strand M13 target (at a final concentration of 20 pM) with Alexa 680-labeled double strand probes (at a final concentration of 50 pM) in 120 mM NaCl, 10 mM Tris pH 8.0, and 5 mM EDTA. The sample to be hybridized was denatured by increasing the pH, followed by neutralization with buffers provided and described in the Label IT Amine Modifying Kit (Mirus, Madison, Wis.). Both samples were incubated for 4 hours at room temperature. Samples were diluted 20-fold into 50 mM Gly Gly pH 8.2 for analysis by single molecule detection for a final concentration of 1 pM target and 2.5 pM probe. Samples were pumped through the instrument's capillary at a rate of 1 ul/min and data was collected for 16 minutes. Both data sets were analyzed for cross-correlation events of 100 photons or greater.

The sample that was denatured and renatured had 564 cross-correlation events of >100 photons per 2 msec in the 16-minute data set analyzed. The control sample that was not denatured yielded only 4 cross-correlation events of >100 photons per 2 msec. The indicate that coincident hybridization-of two fluorescent probes to target can be detected by an the increase in the number of photons given of by a target/probe hybrid molecule as compared to target and probe molecules that are not allowed to form hybrids. The detection of coincident hybridization of two different GV probes to individual target molecules is sufficient to determine a haplotype

The probes used in this experiment just described were large, and it would improve haplotype analysis to use probes that are complementary to shorter sites on the target (8-30 base pairs). Such an assay is described as follows.

The goal is to generate two different 3 kb ssLNA probes (1× average signal intensity), each with affinity to different short loci on ssM13mp18 (equivalent to different GV loci). The additional length of the probes serves as a labeled “tail” for the recognition portion of the probe. By labeling these probe tails with fluorescent dye, we can detect a hybrid-molecule containing ssM13mp18 and both ssLNA probes which produces 2× average fluorescence intensity when analyzed using BioProfile Corporation's single-molecule electrophoresis instrument.

Materials: LNA oligos (Proligo) M13mp18h5594L6017 (200 nm, HPLC 80) 5′-AGG GAA GAA AGC GAA AGG AGG CTG CCA GCG ACG AG (SEQ ID NO: 1); M13mp18h6702L6017 (200 nm, HPLC 80) 5′-AAC CAA TAG GAA CGC CAT CAG CTG CCA GCG ACG AG (SEQ ID NO: 2); Lambda Phage DNA (Sigma cat#D-3654); NovaTaq™ PCR Kit (Novagen cat#71005-3): Deoxynucleotide Triphosphates (10 μmol dNTPs) (Promega cat#U1330); ARES™ Alex Fluor® 680 DNA Labeling Kit (Molecular Probes cat#A-21672); MinElute™ PCR Purification Kit (Qiagen cat#28004); GeneCapsule™ (Geno Technology, Inc.); MJ Research DNA Engine (MJ Research, Inc. cat#PTC-0020, PTC-0225); 8-Strip 0.2 mL Thin-Wall Tubes (MJ Research, Inc. cat#TBS-0201); 8-Strip Caps for 0.2 mL Thin-Wall Tubes (MJ Research, Inc. cat#TCS-0801).

Methods and Results: A PCR reaction mixture is set up to amplify a 4,521base fragment of Lambda Phage DNA using the following components in a 0.2 mL thin-walled thermal cycling tube (final concentrations): Lambda5kLeft oligo (250 nM); Lambda5kRight oligo (250 nM); 10×PCR buffer+MgCl₂(1×); dNTP mix (0.2mM); NovaTaq™ (5 units); Lambda Phage DNA (˜500 ng); Sterile water added to final volume of 50 μL. Samples are thermal cycled for 30 cycles using a standard PCR temperature cycling regiment to generate a PCR amplicon of the predicted size.

The PCR amplicon is analyzed via electrophoresis in a 0.7% agarose gel followed by ethidium bromide staining to verify that the amplicon is the predicted size. The 4,521 base amplicon excised using a GeneCapsule™ and techniques described in the GeneCapsule™ manual. The GeneCapsule™ also removes unincorporated free nucleotides and oligos. The concentration of excised fragment is estimated based on fluorescence of an ethidium bromide stained aliquot of the reaction mixture compared to stained nucleic acids of known concentration.

The purified amplicon is used to generate ss LNA probes. A separate DNA Polymerase extension reaction is set up for each of the LNA oligos by mixing the following components thoroughly in 0.2 mL thin-walled thermal cycling tubes (final concentrations): M13mp18h5594L6017 or M13mp18h6702L6017 (250 nM); 10×PCR buffer+MgCl₂(1×); d[GAC]TP mix (40 μM dGTP, 40 μM dATP, 40 μM dCTP); aminoallyl-dUTP (60 μM); dTTP (10 μM); NovaTaq™ (25 units); 4,521 base Lambda PCR amplicon (500 ng). Sterile water (to final volume of 50 μL). A strip cap is placed on the tube and temperature is cycled using a standard PCR temperature cycling regimen to generate an extension product of ˜3 kb. The extension reaction product is purified using the MinElute™ PCR Purification Kit, following the instructions in the MinElute™ kit manual. An ethanol precipitation step is required after this purification to remove Tris buffer prior to the dye-coupling reaction. Eluted extension product is resuspended in 5 μL nuclease-free water.

The dye-coupling reaction is performed to fluorescently-label the ssLNA probes. Steps 4.1-4.7 in the manual for the ARES™ Alex Fluor® 680 DNA Labeling Kit are followed to couple the dye to the amine-modified probes. Steps 5.1-5.2 in the ARES™ manual describe post-labeling clean up procedures, which include another MinElute™ PCR Purification column and ethanol precipitation.

The labeled ssLNA probes is resuspended in 50 mM Gly-Gly buffer pH 8.2 and diluted to a final concentration of 100 pM for each probe. A small volume of each probe is aliquoted into a separate pre-siliconized 1.5 mL tube and diluted further to a final concentration of 10-50 fM in 50 mM Gly-Gly buffer pH 8.2. These dilutions are analyzed on the single molecule electrophoresis instrument to verify the following: the ability to detect each probe individually, the relative average intensity of each ssLNA probe, and to determine the actual concentration of each probe.

A hybridization reaction is initiated containing both ssLNA probes and ssM13mp18 DNA. The following components are mixed in a pre-siliconized 1.5 mL tube (final concentrations) M13mp18h5594L6017 (10 pM); M13mp18h6702L6017 (10 pM); ssM13mp18 DNA (1 pM); pre-filtered hybridization buffer (5×SSC, 0.1% N-Laroylsarcosine, 0.02% SDS), sterile water (to final volume of 100 uL). The hybridization reaction is incubated at 65° C. for up to 16 hours. A small is aliquoted to a new pre-siliconized 1.5 mL tube and dilute 1:100 in 50 mM Gly-Gly buffer pH 8.2 to a final ssM13mp18 template concentration of ˜10 fM. These dilutions are analyzed using the single-molecule electrophoresis instrument.

Populations of molecules with 1× and 2× average signal intensities are detected. 1× average signal intensity molecules represent unhybridized ssLNA probe and LNA probes which are still bound to the Lambda fragment following the extension reaction. Molecules with 2× signal intensity represent hybrid of both probes to ssM13mp18 target. Such an analysis of GVs using two probes is sufficient to determine the haplotype of the target.

Other Embodiments

The invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

References Cited

All references cited above are incorporated herein by reference in their entirety and for all purposes to the same extent as if each individual publication, patent or patent application was specifically and individually indicated to be incorporated by reference in its entirety for all purposes. Citation of a reference herein shall not be construed as an admission that such is prior art to the present invention.

What is claimed is:

1. A method for determining genetic haplotype comprising:
(a) identifying a target nucleic acid molecule or gene fragment, said nucleic acid molecule or gene fragment comprising a haplotype of interest, by:
(i) hybridizing a primer recognizing a first genetic variant, said variant correlating to a haplotype, to the target nucleic acid molecule or gene fragment and wherein a labeled primer dependent transcript is generated from the target nucleic acid or gene fragment; and
(1) hybridizing at least one labeled probe, said probe recognizing a second genetic variant downstream from the primer, to the primer dependent transcript, or
(2) hybridizing at least one unlabeled probe, said probe recognizing recognizing a second genetic variant downstream from the primer, to the primer dependent transcript; and
(b) detecting at least one parameter displayed by one of the primer-dependent transcript, the at least one probe, or a primer-dependent transcript/probe complex, thereby correlating the displayed parameter to the haplotype.

2. The method of claim 1, wherein the parameter displayed is selected from the group consisting of fluorescence lifetime, fluorescence polarization, mass, net charge, shape, diffusion, electrophoretic velocity, fluorescence, fluorescence intensity, luminescence, luminescence intensity, chemiluminescence, chemiluminescence intensity, light absorption, electrical reactance, and cooperative hybridization.

3. The method of claim 1, wherein the parameter of step (b) is displayed in an electric field.

4. The method of claim 1, wherein the labeled nucleic acid or gene fragment is separated from unlabeled nucleic acids prior to detection step (b).

5. The method of claim 4, wherein the labeled nucleic acid or gene fragment is separated from unlabeled nucleic acids by a change in the charge, mass or shape of the labeled nucleic acid in an electric field.

6. The method of claim 4, wherein the labeled nucleic acid or gene fragment is separated from unlabeled nucleic acids by sieving the labeled nucleic acid in an electric field.

7. The method of claim 4, wherein the labeled nucleic acid or gene fragment is distinguished from unlabeled nucleic acids by a change in the charge, mass or shape of the labeled nucleic acid in an electric field.

8. The method of claim 4, wherein the labeled nucleic acid or gene fragment is distinguished from unlabeled nucleic acids by sieving the labeled nucleic acid in an electric field.

9. The method of claim 1, wherein the probe is bound to a molecule selected from the group consisting of a microsphere, nanosphere, barcode particle, and nanocrystal.

10. The method of claim 1, wherein the probe is selected from the group consisting of a nucleic acid, oligonucleotide, peptide, polypeptide, lipid, sterol, biological molecule, dye, drug, mass tag, isotope, and any combination thereof.

11. The method of claim 10, wherein the nucleic acid is selected from the group consisting of PNA, LNA, DNA, RNA, and DNX.

12. The method of claim 1, wherein the probe is bound to the nucleic acid or gene fragment covalently.

13. The method of claim 1, wherein the probe is in communication with:
(a) at least one nucleic acid or gene fragment; and
(b) at least one solid substrate.

14. A method for determining genetic haplotype comprising:
(a) identifying a target nucleic acid molecule or gene fragment, said nucleic acid molecule or gene fragment comprising a haplotype of interest, by:
(i) hybridizing a primer recognizing a first genetic variant, said variant correlating to a haplotype, to the target nucleic acid molecule or gene fragment and wherein a primer dependent transcript is generated from the target nucleic acid or gene fragment; and
(1) hybridizing at least one labeled probe, said probe recognizing a second genetic variant downstream from the primer, to the primer dependent transcript, or
(2) hybridizing at least one unlabeled probe, said probe recognizing a second genetic variant downstream from the primer, to the labeled primer dependent transcript; and
(b) detecting at least one parameter displayed by one of the labeled primer-dependent transcript, the at least one probe, or a primer-dependent transcript/probe complex, thereby correlating the displayed parameter to the haplotype.

15. The method of claim 14, wherein the parameter displayed is selected from the group consisting of fluorescence lifetime, fluorescence polarization, mass, net charge, shape, diffusion, electrophoretic velocity, fluorescence, fluorescence intensity, luminescence, luminescence intensity, chemiluminescence, chemiluminescence intensity, light absorption, electrical reactance, and cooperative hybridization.

16. The method of claim 14, wherein the parameter of step (b) is displayed in an electric field.

17. The method of claim 14, wherein the labeled nucleic acid or gene fragment is separated from unlabeled nucleic acids prior to detection step (b).

18. The method of claim 17, wherein the labeled nucleic acid or gene fragment is separated from unlabeled nucleic acids by a change in the charge, mass or shape of the labeled nucleic acid in an electric field.

19. The method of claim 17, wherein the labeled nucleic acid or gene fragment is separated from unlabeled nucleic acids by sieving the labeled nucleic acid in an electric field.

20. The method of claim 17, wherein the labeled nucleic acid or gene fragment is distinguished from unlabeled nucleic acids by a change in the charge, mass or shape of the labeled nucleic acid in an electric field.

21. The method of claim 17, wherein the labeled nucleic acid or gene fragment is distinguished from unlabeled nucleic acids by sieving the labeled nucleic acid in an electric field.

22. The method of claim 14, wherein the probe is bound to a molecule selected from the group consisting of a microsphere, nanosphere, barcode particle, and nanocrystal.

23. The method of claim 14, wherein the probe is selected from the group consisting of a nucleic acid, oligonucleotide, peptide, polypeptide, lipid, sterol, biological molecule, dye, drug, mass tag, isotope, and any combination thereof.

24. The method of claim 23, wherein the nucleic acid is selected from the group consisting of PNA, LNA, DNA, RNA, and DNX.

25. The method of claim 14, wherein the probe is bound to the nucleic acid or gene fragment covalently.

26. The method of claim 14, wherein the probe is in communication with:
(a) at least one nucleic acid or gene fragment; and
(b) at least one solid substrate.

27. A method for determining genetic haplotype comprising:
(a) labeling a nucleic acid molecule or gene fragment with at least two probes, each probe recognizing a different genetic variation that defines a haplotype; and
(b) detecting the nucleic acid molecule or gene fragment by measuring a sequential change in a single parameter displayed by the probes, thereby rendering the genetic haplotype determinable.

28. The method of claim 27, wherein the parameter displayed is selected from the group consisting of fluorescence lifetime, fluorescence polarization, mass, net charge, shape, diffusion, electrophoretic velocity, fluorescence, luminescence, chemiluminescence, light absorption, electrical reactance, and cooperative hybridization.

29. The method of claim 27, wherein the parameter of step (b) is displayed in an electric field.

30. The method of claim 27, wherein the labeled nucleic acid or gene fragment is separated from unlabeled nucleic acids prior to detection step (b).

31. The method of claim 30, wherein the labeled nucleic acid or gene fragment is separated from unlabeled nucleic acids by a change in the charge, mass or shape of the labeled nucleic acid in an electric field.

32. The method of claim 30, wherein the labeled nucleic acid or gene fragment is separated from unlabeled nucleic acids by sieving the labeled nucleic acid in an electric field.

33. The method of claim 30, wherein the labeled nucleic acid or gene fragment is distinguished from unlabeled nucleic acids by a change in the charge, mass or shape of the labeled nucleic acid in an electric field.

34. The method of claim 30, wherein the labeled nucleic acid or gene fragment is distinguished from unlabeled nucleic acids by sieving the labeled nucleic acid in an electric field.

35. The method of claim 27, wherein the probe is bound to a molecule selected from the group consisting of a microsphere, nanosphere, barcode particle, and nanocrystal.

36. The method of claim 27, wherein the probe is selected from the group consisting of a nucleic acid, oligonucleotide, peptide, polypeptide, lipid, sterol, biological molecule, dye, drug, mass tag, isotope, and any combination thereof.

37. The method of claim 36, wherein the nucleic acid is selected from the group consisting of PNA, LNA and DNX.

38. The method of claim 27, wherein the probe is bound to the nucleic acid or gene fragment covalently.

39. The method of claim 27, wherein the probe is in communication with:
(a) at least one nucleic acid or gene fragment; and
(b) at least one solid substrate.

40. A method for determining genetic haplotype comprising:
(a) labeling a nucleic acid molecule or gene fragment with at least two probes, each probe recognizing a different genetic variation that defines a haplotype; and
(b) detecting the nucleic acid molecule or gene fragment by measuring a sequential change in at least two parameters displayed by the probes, thereby rendering the genetic haplotype determinable.

41. The method of claim 40, wherein the parameters displayed are selected from the group consisting of fluorescence lifetime, fluorescence polarization, mass, net charge, shape, diffusion, electrophoretic velocity, fluorescence, luminescence, chemiluminescence, light absorption, electrical reactance, cooperative hybridization, and any combination thereof.

42. The method of claim 40, wherein the parameters of step (b) are displayed in an electric field.

43. The method of claim 40, wherein the labeled nucleic acid or gene fragment is separated from unlabeled nucleic acids prior to detection step (b).

44. The method of claim 43, wherein the labeled nucleic acid or gene fragment is separated from unlabeled nucleic acids by a change in the charge, mass or shape of the labeled nucleic acid in an electric field.

45. The method of claim 43, wherein the labeled nucleic acid or gene fragment is separated from unlabeled nucleic acids by sieving the labeled nucleic acid in an electric field.

46. The method of claim 43, wherein the labeled nucleic acid or gene fragment is distinguished from unlabeled nucleic acids by a change in the charge, mass or shape of the labeled nucleic acid in an electric field.

47. The method of claim 43, wherein the labeled nucleic acid or gene fragment is distinguished from unlabeled nucleic acids by sieving the labeled nucleic acid in an electric field.

48. The method of claim 40, wherein the probe is bound to a molecule selected from the group consisting of a microsphere, nanosphere, barcode particle, and nanocrystal.

49. The method of claim 40, wherein the probe is selected from the group consisting of a nucleic acid, oligonucleotide, peptide, polypeptide, lipid, sterol, biological molecule, dye, drug, mass tag, isotope, and any combination thereof.

50. The method of claim 49, wherein the nucleic acid is selected from the group consisting of PNA, LNA and DNX.

51. The method of claim 40, wherein the probe is bound to the nucleic acid or gene fragment covalently.

52. The method of claim 40, wherein the probe is in communication with:
(a) at least one nucleic acid or gene fragment; and
(b) at least one solid substrate.

53. A method for determining genetic haplotype comprising:
(a) labeling a nucleic acid molecule or gene fragment with at least two probes, each probe recognizing a different genetic variation that defines a haplotype; and
(b) detecting the nucleic acid molecule or gene fragment by simultaneously measuring the parameters displayed by the probes, wherein the parameter is not cooperative hybridization, thereby rendering the genetic haplotype determinable.

54. The method of claim 53, wherein the parameter displayed is selected from the group consisting of fluorescence lifetime, fluorescence polarization, mass, net charge, shape, diffusion, electrophoretic velocity, fluorescence, luminescence, chemiluminescence, light absorption, and electrical reactance.

55. The method of claim 53, wherein the parameter of step (b) is displayed in an electric field.

56. The method of claim 53, wherein the labeled nucleic acid or gene fragment is separated from unlabeled nucleic acids prior to detection step (b).

57. The method of claim 56, wherein the labeled nucleic acid or gene fragment is separated from unlabeled nucleic acids by a change in the charge, mass or shape of the labeled nucleic acid in an electric field.

58. The method of claim 56, wherein the labeled nucleic acid or gene fragment is separated from unlabeled nucleic acids by sieving the labeled nucleic acid in an electric field.

59. The method of claim 56, wherein the labeled nucleic acid or gene fragment is distinguished from unlabeled nucleic acids by a change in the charge, mass or shape of the labeled nucleic acid in an electric field.

60. The method of claim 56, wherein the labeled nucleic acid or gene fragment is distinguished from unlabeled nucleic acids by sieving the labeled nucleic acid in an electric field.

61. The method of claim 53, wherein the probe is bound to a molecule selected from the group consisting of a microsphere, nanosphere, barcode particle, and nanocrystal.

62. The method of claim 53, wherein the probe is selected from the group consisting of a nucleic acid, oligonucleotide, peptide, polypeptide, lipid, sterol, biological molecule, dye, drug, mass tag, isotope, and any combination thereof.

63. The method of claim 62, wherein the nucleic acid is selected from the group consisting of PNA, LNA and DNX.

64. The method of claim 53, wherein the probe is bound to the nucleic acid or gene fragment covalently.

65. The method of claim 53, wherein the probe is in communication with:
(a) at least one nucleic acid or gene fragment; and
(b) at least one solid substrate.

66. A method for determining genetic haplotype comprising:
(a) labeling a nucleic acid molecule or gene fragment with at least two probes, each probe recognizing a different genetic variation that defines a haplotype; and
(b) detecting the nucleic acid molecule or gene fragment by simultaneously measuring at least two parameters displayed by the probes, wherein the parameter is not cooperative hybridization, and further wherein the probe is bound to a molecule selected from the group consisting of a microsphere, nanosphere and bar code particle, thereby rendering the genetic haplotype determinable.

67. The method of claim 66, wherein the parameter displayed is selected from the group consisting of fluorescence lifetime, fluorescence polarization, mass, net charge, shape, diffusion, electrophoretic velocity, fluorescence, luminescence, chemiluminescence, light absorption, and electrical reactance.

68. The method of claim 66, wherein the parameter of step (b) is displayed in an electric field.

69. The method of claim 66, wherein the labeled nucleic acid or gene fragment is separated from unlabeled nucleic acids prior to detection step (b).

70. The method of claim 69, wherein the labeled nucleic acid or gene fragment is separated from unlabeled nucleic acids by a change in the charge, mass or shape of the labeled nucleic acid in an electric field.

71. The method of claim 69, wherein the labeled nucleic acid or gene fragment is separated from unlabeled nucleic acids by sieving the labeled nucleic acid in an electric field.

72. The method of claim 69, wherein the labeled nucleic acid or gene fragment is distinguished from unlabeled nucleic acids by a change in the charge, mass or shape of the labeled nucleic acid in an electric field.

73. The method of claim 69, wherein the labeled nucleic acid or gene fragment is distinguished from unlabeled nucleic acids by sieving the labeled nucleic acid in an electric field.

74. The method of claim 66, wherein the probe is bound to a molecule selected from the group consisting of a microsphere, nanosphere, barcode particle, and nanocrystal.

75. The method of claim 66, wherein the probe is selected from the group consisting of a nucleic acid, oligonucleotide, peptide, polypeptide, lipid, sterol, biological molecule, dye, drug, mass tag, isotope, and any combination thereof.

76. The method of claim 75, wherein the nucleic acid is selected from the group consisting of PNA, LNA and DNX.

77. The method of claim 66, wherein the probe is bound to the nucleic acid or gene fragment covalently.

78. The method of claim 66, wherein the probe is in communication with:
(a) at least two nucleic acids or gene fragments; and
(b) at least two solid substrates.

79. A method for determining genetic haplotype comprising:
(a) labeling a nucleic acid molecule or gene fragment with at least one probe, each probe recognizing a different genetic variation that defines a haplotype; and
(b) detecting the velocity of the nucleic acid molecule or gene fragment by measuring the difference in time the probe displays a parameter measured by a first detector at a first position and a second detector at a second position, wherein the probe is bound to a molecule selected from the group consisting of a microsphere, nanosphere, bar code particle, and nanocrystal, thereby rendering the genetic haplotype determinable.

80. The method of claim 79, wherein the velocity is visually detected on a CCD detector.

81. A method for determining genetic haplotype comprising:
(a) labeling a nucleic acid molecule or gene fragment with a first probe and a second probe, said first probe and second probe recognizing a different genetic variation that defines a haplotype; and
(b) detecting the velocity of the nucleic acid molecule or gene fragment by sequentially measuring a first parameter displayed by the first probe and a second parameter displayed by the second probe, wherein the first or second probe is bound to a molecule selected from the group consisting of a microsphere, nanosphere, bar code particle, and nanocrystal, thereby rendering the genetic haplotype determinable.

82. The method of claim 81, wherein the velocity is visually detected on a CCD detector.
